(19) United States
(12) Patent Application Publication
(10) Pub. No.: US 2004/0013729 A1
(43) Pub. Date: Jan. 22, 2004

(54) Single-drop multiple-agent composition for topical delivery to the eye

(76) Inventor: Lawrence M. Buono, Philadelphia, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/197,784

(22) Filed: Jul. 18, 2002

(51) Int. Cl. A61K 9/14; A61K 31/137

(52) U.S. Cl. 424/486; 514/649

(57) ABSTRACT

A composition for topical use on an eye, for example comprising a mydriatic agent, a cycloplegic agent, a topical anesthetic, and a viscoelastic polymer; also methods of topically administering such compositions to the eye as part of diagnostic and therapeutic procedures.

### BACKGROUND OF THE INVENTION

The present invention relates to ophthalmic agent compositions, especially ones comprising mydriatic and cycloplegic agents.

In the course of ophthalmic practice, it is often necessary to dilate the pupil in order to facilitate several diagnostic and therapeutic endeavors. Such dilation is typically facilitated by the use of topical mydriatic and cycloplegic agents. The barrier to delivering those agents to their intended target is the cornea. The cornea has several histologic layers, one of which is the corneal epithelium. This layer is primarily responsible for the barrier. Agents intended for dilation must penetrate this barrier so as to enter the anterior chamber and reach their target tissue, namely the iris sphincter, iris dilator and ciliary body. This is achieved by the contact of the agent with the corneal surface. The longer the contact of the agent with the corneal surface, the greater the possibility that the agent will penetrate that surface, hence reaching the target tissue.

Currently, when a mydriatic agent and a cycloplegic agent are applied to the corneal surface in successive drops, the time that each agent is on the surface is limited by several factors. The natural blinking of the eyelid easily washes the agent away. Also, if the second drop is too rapidly instilled after the first one, the first drop tends to be washed away. Both of these effects serve to diminish the duration of which the agents contact the ocular surface, hence decreasing the amount of agent available to reach the target tissue

### BRIEF SUMMARY OF THE INVENTION

In a general aspect, the invention is a composition for topical use on an eye, especially a human eye. The topical composition comprises:

an amount of one or more mydriatric agents sufficient to cause mydriasis;

an amount of one or more cycloplegic agents sufficient to cause cycloplegic;

a viscoelastic polymer; and

water.

The viscoelastic polymer is important to the invention, as its presence increases the time available for the agents to contact the cornea. The result is greater agent penetration and delivery to the intended target tissue.

In particular embodiments, the topical composition further comprises an amount of one or more anesthetics sufficient to cause anesthesia in the eye and/or an amount of one or more non-steroidal anti-inflammatory agents.

A method of inducing iris dilation, where the method comprises administering such a topical composition, is also an aspect of the present invention. Examples of situations where the method is employed include eye examinations as part of diagnostic procedures, and during intraocular surgery or laser surgery.

The major advantage of the present invention is that it is only necessary to administer a single drop, as opposed to two or three drops. (Although 2 or more drops can be administered if desired.) This is a benefit to both the physician and the patient as it saves time and decreases discomfort to the patient. Furthermore, increased pupillary size often allows therapeutic and surgical procedures to be achieved in a safer manner than if the pupil is not maximally dilated.

### DETAILED DESCRIPTION OF THE INVENTION

Mydriatic agents are sympathomimetic agents that, in pharmaceutically effective amounts, cause iris dilation, especially as a result of excitation of the iris dilator muscle.

Mydriatic agents suitable for the present invention include, but are not limited to, phenylephrine, naphazoline, and epinephrine. A pharmaceutically effective amount of a mydriatic agent is one that will cause, in an adult after 30 minutes, an increase in the diameter of the iris of at least 10 percent.

Cycloplegic agents are parasympatholytic agents that, in pharmaceutically effective amounts, cause cycloplegic, especially as a result of paralysis of both the ciliary muscle for accommodation and the iris sphincter muscle. Cycloplegic agents suitable for the present invention include, but are not limited to, tropicamide, cyclopentolate, scopolomine, homatropine, and atropine. A pharmaceutically effective amount of a cycloplegic agent is an amount that will result in detectable blurring of vision in an adult. A pharmaceutically effective amount of a cycloplegic agent is one that will cause, in an adult after 30 minutes, a detectable blurring of vision and mydriasis.

A pharmaceutically effective amount of an anesthetic is one that will cause a noticeable decrease in pain for an adult during the administration of a drop of a composition of the present invention. In preferred embodiments, the anesthetic also facilitates penetration of the mydriatic and cycloplegic agents into the cornea. Examples of the many possible anesthetics that can be used include proparacaine, bupivicaine, and tetracaine.

The viscoelastic polymer is present in sufficient amounts to make the composition viscous. A highly preferred viscoelastic polymer is hydroxypropylmethylcellulose (HPMC). HPMC is relatively inexpensive but nevertheless effective. A 2.5% solution of that polymer is manufactured by CIBA Vision Corporation under the name Goniosol®. That solution was used to make the compositions tested as described in the Examples below. A highly preferred final concentration of the HPMC in the composition of the invention is 1.0%. Generally, it should be at least 0.1%. It is understood that the viscosity of a solution with a given concentration of HPMC can vary from manufacturer to manufacturer (and possibly from batch to batch from a given manufacturer). A guideline for adjusting the concentration of a particular HPMC in a solution, or that of another viscoelastic polymer, is that a 1.0% concentration in the composition of the invention is the presumptively correct concentration but the dilation obtained with such a 1.0% concentration should be at least as good as that described below in the examples. Should the results not be as good, the viscosity of the viscoelastic polymer can be varied to determine its satisfactory concentration and that concentration can be used in other compositions that have the same HPMC.

Other viscoelastic polymers can be substituted for HPMC. They include but are not limited to propylmethylcellulose, methylcellulose, carboxymethylcellulose, hyaluronate, and chondroitin sulfate.

A non-steroidal anti-inflammatory drug (“NSAID”) is a non-steroidal agent that, when administered in a pharmaceutically effective amounts, decreases the amount of tissue inflammation (e.g., redness, swelling, pain). Examples of the many non-steroidal anti-inflammatory agents are flubiprofen, ketorolactromethamine, and diclofenac sodium (Voltaren). In preferred embodiments, the NSAID facilitates dilation.

Example of some of the preferred compositions of the invention are the following Compositions A, B, and C. For each composition, the concentrations of each agent in the composition is indicated.

Composition A:

proparacaine, 0 05%

phenylephrine, 0.63%

tropicamide, 0.25%

hydroxypropylmethylcellulose, 1.0%.

Composition A is well-adapted for use in an ophthamologist's office for purposes of eye examination of an adult. It can be used without the proparacaine if desired.

Composition B:

proparacaine, 0.05%

phenylephrine, 0.63%

tropicamide, 0.2%

cyclopentolate, 0.1%

hydroxypropylmethylcellulose, 1.0%

Composition B is well-adapted to achieve iris dilation as part of a surgical procedure or for pediatric examination.

Composition C:

proparacaine, 0.045%

flurbiprofin, 0.0012%

phenylephrine, 0.48%

tropicamide, 0.19%

cyclopentolate, 0.09%

hydroxypropylmethylcellulose, 1.0%

Composition C, like Composition B, is well adapted for achieving iris dilation as part of a surgical procedure.

Compositions can, for example, be made from commercially available solutions of the various components, which solutions are available as follows (concentrations in parenthesis): proparacaine (0.5%), phenylephrine (2.5%), tropicamide (1%), cyclopentalate (1%), flurbiprofen (0.3%). These available solutions are typical ones for administering single drops of the respective reagents as is current ophthalmologic practice. They come in solutions of physiologically acceptable pH and therefore their combination so as to form Composition A also has a physiologically acceptable pH. It is noteworthy that these stock solutions all are diluted for purposes of making Compositions A, B, and C. Therefore alternative preferred compositions of the invention include one or more of the agents at the same concentration that they appear in the above-noted commercially available solutions. This is particularly true of the mydriatic and cycloplegic reagents. As a result, Composition A, for example, could be modified to include 2.5% phenylephrine and/or 1% tropicamide.

The following examples is intended to illustrate the invention rather than limit it.

### EXAMPLES

### Example 1

The effect on iris dilation of administering a single drop of Composition A was compared to the effect of a 3-drop control protocol.

In each of 8 adult subjects, a 3-drop control protocol reflective of the prior art was performed with one eye and a single drop protocol using Composition A of the present invention was administered to the other eye.

The control protocol involved administration of 3 successive drops: proparacaine (0.5%), phenylephrine (2.5%), and tropicamide (1%). There were intervals of about 15 seconds between administration of the proparacaine and phenylephrine drops, and also between administration of the phenylephrine and tropicamide drops.

Within 5 seconds of the time that tropicamide was administered to the first eye, a single drop of Composition A was administered to the second eye The bottle dispenser used to administer Composition A was of the same type as used for the drops in the control protocol.

The results are shown in Tables 1-4 The time in the left-most column in Tables 1-4 was the time interval since the administration of Composition A.

Measurements of pupil diameter were made to the closest 0.5 mm at 3 minute intervals. The results for 0, 9, 15, 21, 30, 45, and 60 minutes are summarized in the Tables.

The results in Table 1 represents averages for all treated persons. Those in Table 2 represent averages for the 4 subjects with blue eyes. Those in Table 3 represent the averages for the 4 subjects with brown (3) or hazel (1) eyes.

TABLE 1
Average size of pupils of the eyes tested

| | Composition A | Control Protocol | |
|---|---|---|---|
| Time (minutes) | (mm) | (mm) | Difference (mm) |
| 0 | 4.2 | 4.2 | 0 |
| 9 | 4.5 | 4.3 | 0.2 |
| 15 | 5.0 | 4.6 | 0.4 |
| 21 | 5.6 | 4.9 | 0.7 |
| 30 | 6.7 | 5.5 | 1.2 |
| 45 | 8.1 | 7.3 | 0.8 |
| 60 | 8.2 | 7.8 | 0.4 |

TABLE 2
Average size of pupils of blue eyes

| | Composition A | Control Protocol | |
|---|---|---|---|
| Time (minutes) | (mm) | (mm) | Difference (mm) |
| 0 | 4.6 | 4.6 | 0 |
| 9 | 4.9 | 46 | 0.3 |
| 15 | 5.4 | 4.9 | 0.5 |
| 21 | 5.9 | 5.4 | 0.4 |
| 30 | 7.1 | 5.8 | 1.3 |
| 45 | 8.6 | 7.4 | 1.2 |
| 60 | 8.8 | 8.0 | 0.8 |

TABLE 3
Average size of pupils of brown/hazel eyes

| | Composition A | Control Protocol | |
|---|---|---|---|
| Time (minutes) | (mm) | (mm) | Difference (mm) |
| 0 | 3.9 | 3.9 | 0 |
| 9 | 4.1 | 4 | 0.1 |
| 15 | 4.6 | 4.3 | 0.3 |
| 21 | 5.3 | 4.8 | 0.5 |
| 30 | 6.3 | 5.3 | 1.0 |
| 45 | 7.5 | 7.3 | 0.2 |
| 60 | 7.6 | 7.5 | 0.1 |

Table 4 summarizes differences in the amount of dilation caused by the single drop composition of the invention as compared to that caused by the 3-drop control protocol. The differences were calculated separately for the data in Tables 1-3, respectively, and summarized in Table 4. All the percentages in Table 4 reflect the extent to which the single drop method of the invention caused more dilation than the 3-drop protocol of the prior art.

TABLE 4
Difference in the increase of dilation caused by composition A
and that caused by the control protocol, expressed as a
percentage of the initial iris diameter

| Time (minutes) | All Eyes (%) | Blue (%) | Brown/Hazel (%) |
|---|---|---|---|
| 0 | 0 | 0 | 0 |
| 9 | 4.8 | 6.5 | 2.6 |
| 15 | 9.5 | 10.8 | 7.7 |
| 21 | 16.7 | 8.7 | 12.8 |
| 30 | 28.6 | 28.3 | 25.6 |
| 60 | 9.4 | 17.4 | 2.6 |

In Table 4, the “All Eyes” column refers to results averaged for all 8 subjects. It can be seen that Composition A drops dilated All Eyes more quickly on the average than the control protocol did and achieved a greater size of dilation than the control protocol did.

The results in Tables 1-4 represent average values. Nevertheless, there was a high degree of consistency in the results achieved. For each of the persons tested, the dilation achieved after 30 minutes with a single drop of composition A was greater than that with the 3-drop control protocol. For 5 persons, the dilation achieved after 60 minutes was greater than that achieved with the control protocol and for 3 persons it was the same after 60 minutes.

An analysis of the data different from that described above also showed the superiority of the single drop method. It was calculated that the composition A drops took an average of 23.4 minutes to achieve 6.0 mm of dilation, whereas the regular drops took 31.9 minutes to achieve 6.0 mm of dilation, a difference of 8.5 minutes. For blue eyes, this difference was even greater, with the average for the composition A drops being 19.5 minutes and the average for the control protocol drops being 28.5 minutes, a difference of 9 minutes. Similarly, with All Eyes, the time for composition A drops to achieve 7.0 mm dilation was only 78% of the time required by the control protocol. With blue eyes, the time for composition A drops to achieve 7.0 mm dilation was only 68% of the time required by the control protocol.

It is noteworthy that Composition A was made by diluting stock solutions of phenylephrine and tropicamide whereas in the control protocol, undiluted stock solutions were used. As a result, the amounts of phenylephrine and tropicamide in the compositon A drops were several times lower in concentration than their amounts in the control protocol drops. Nevertheless, superior results were still obtained with the current invention as represented by Composition A.

1. A composition for topical administration to the eye, said composition comprising:
an amount of one or more mydriatric agents sufficient to cause mydriasis;
an amount of one or more cycloplegic agents sufficient to cause cycloplegic;
a viscoelastic polymer; and
water.

2. A composition of claim 1 further comprising an amount of one or more anesthetics sufficient to cause anesthesia in the eye.

3. A composition of claim 2 that also facilitates drug penetration.

4. A composition of claim 1 further comprising an amount of one or more non-steroidal anti-inflammatory agents.

5. A composition of claim 4 further comprising an amount of one or more anesthetics sufficient to cause anesthesia in the eye.

6. A composition of claim 1 comprising a mydriatic agent selected from the group consisting of phenylephrine, naphazoline, and epinephrine.

7. A composition of claim 6 wherein the mydriatic agent is phenylephrine.

8. A composition of claim 1 comprising a cycloplegic agent selected from the group consisting of tropicamide, cyclopentolate, scopolomine, homatropine, and atropine.

9. A composition of claim 8 wherein the cycloplegic agent is tropicamide.

10. A composition of claim 2 comprising an anesthetic selected from the group consisting of proparacaine, bupivicaine, and tetracaine

11. A composition of claim 1 comprising a viscoelastic polymer selected from the group consisting of hydroxypropylmethylcellulose, propylmethylcellulose, methylcellulose, carboxymethylcellulose, hyaluronate, and chondroitin sulfate.

12. A composition of claim 11 wherein the viscoelastic polymer is hydroxypropylmethylcellulose.

13. A composition of claim 1 comprising phenylephrine and tropicamide,

14. A composition of claim 13 further comprising hydroxypropylmethylcellulose.

15. A composition of claim 14 comprising a non-steroidal anti-inflammatory agent.

16. A method of inducing iris dilation in an eye, said method comprising administering a composition to the surface of an eye, said composition comprising
an amount of one or more mydriatric agents sufficient to cause mydriasis;
an amount of one or more cycloplegic agents sufficient to cause cycloplegic;
a viscoelastic polymer; and
water.

17. A method of claim 16 wherein the composition further comprises an amount of one or more anesthetics sufficient to cause anesthesia in the eye.

18. A method of claim 17 wherein the composition further comprises an amount of one or more non-steroidal anti-inflammatory agents.
